(19) United States
(12) Plant Patent Application Publication
(10) Pub. No.: US 2003/0182698 P1
(43) Pub. Date: Sep. 25, 2003

(54) Chinese kousa Dogwood named 'Elsbry'

(76) Inventor: John E. Elsley, Greenwood, SC (US)

Correspondence Address: McNAIR LAW FIRM, P.A., P.O. Box 10827, Greenville, SC, 29603-0827, US

(21) Appl. No.: 10/103,003

(22) Filed: Mar. 21, 2002

(51) Int. Cl. A01H 5/00

(52) U.S. Cl. PLT/220

(57) ABSTRACT

A new and distinct cultivar of evergreen Dogwood tree named ‘Elsbry’ that is particularly characterized by the unique combination of its vigorous growth rate, flowering at a young age, prolific flower production on an annual basis, blooms 2-3 weeks later and over a longer period than other Cornus kousa selections, fruits extend ornamental interest into the early fall season, increased disease resistance, and good cold hardiness.

### FIELD OF THE INVENTION

The present invention relates to a new and distinct cultivar of the evergreen variety of the native Chinese Kousa Dogwood, botanically known as Cornus kousa of the variety angustata Chun 1934, a plant since classified as a distinct species Cornus angustata (Chun) T. R. Dudley (Phytologia. Vol. 76, pg. 428, 1994) in 1994 by Dr. T. R. Dudley of the United States National Arboretum, Washington, D.C. This new cultivar of Dogwood is hereinafter referred to by the cultivar name ‘Elsbry’.

### BACKGROUND OF THE INVENTION

The new Dogwood was discovered by the Inventor in 1993 as a 2-3 year old chance seedling at a nursery in Greenwood, S.C. Its previous origin is unknown, but it is believed the seedling may have originated from seed collected in China. The seedling was planted and cultivated at the home of the Inventor in Greenwood, S.C., where it was discovered and recognized to be a new and distinct variety of Dogwood having the unique characteristics described herein below. Asexual reproduction of the new Dogwood by grafting has shown that the unique features associated with the plant are stable and reproduced true to type in successive generations.

### SUMMARY OF THE INVENTION

Plants of the new ‘Elsbry’ Dogwood have not been observed under all possible environmental conditions. Accordingly, the phenotype may vary somewhat with environmental variations such as temperature, day length, light intensity, nutrition and water status without, however, any variance in genotype.

The following traits have been repeatedly observed and are determined to be the unique characteristics of ‘Elsbry’. The characteristics in combination distinguish ‘Elsbry’ as a new and distinct cultivar of Cornus angustata:

1. Vigorous growth rate.

2. Flowers produced at a young age, specifically, on 2 year old grafted plants.

3. Prolific flower production on an annual basis.

4. Blooms 2-3 weeks later and over a longer period than other Cornus kousa selections. In Greenwood, S.C., flowering normally commences in mid May and finishes in late June.

5. Fruits extend ornamental interest into the early fall season.

6. The evergreen foliage displays resistance to powdery mildew and leaf and stem anthracnose, two major disease problems with many Dogwood varieties.

7. Exhibits good cold hardiness having withstood 6° F. temperatures. Probable hardiness range zone being from 6-10 on the U.S. Department of Agriculture Plant Hardiness Zone Map (1990).

### BRIEF DESCRIPTION OF THE PHOTOGRAPHS

The accompanying colored photographs illustrate the overall appearance of the new ‘Elsbry’ Dogwood showing the colors as true as it is reasonably possible to obtain in colored reproduction of this type.

FIG. 1, shows a typical specimen of the entire ‘Elsbry’ Dogwood in full bloom;

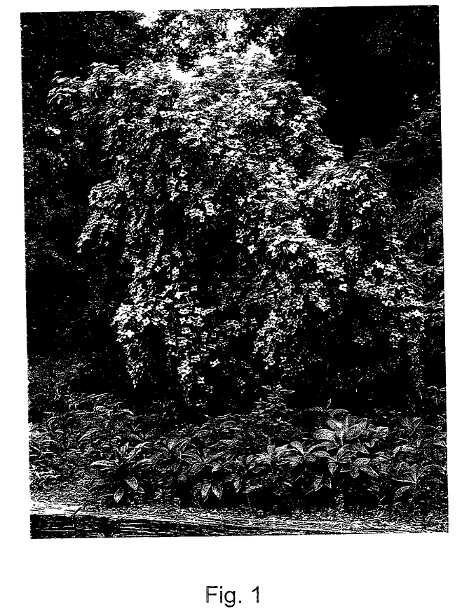

FIG. 2, shows a detailed close up view of the flowers and foliage;

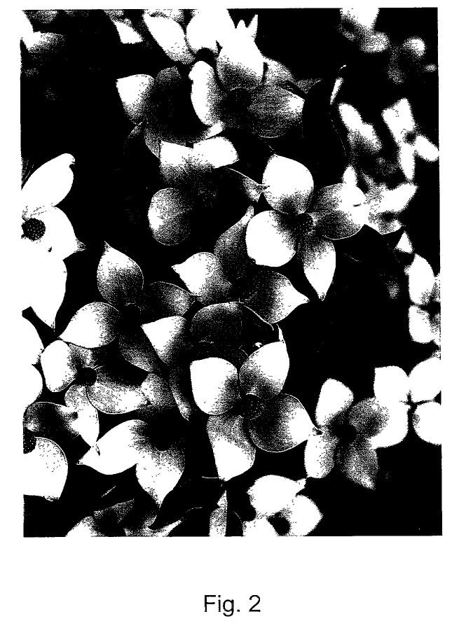

FIG. 3, shows the fruits typically produced in the month of September; and,

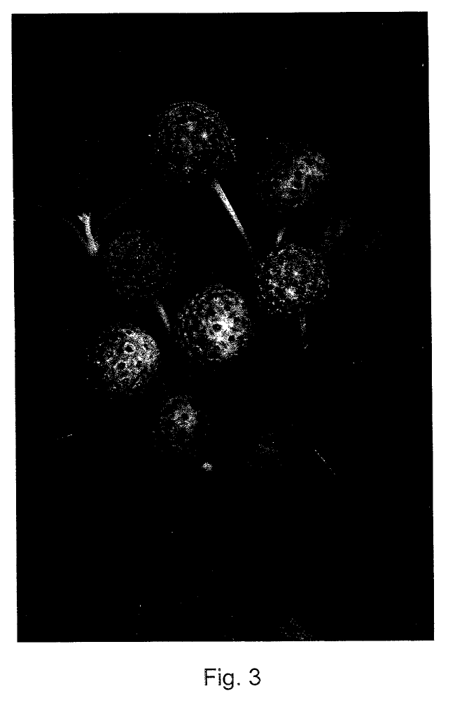

FIG. 4, shows several leaves against a ruler to illustrate the foliage characteristics and dimensions.

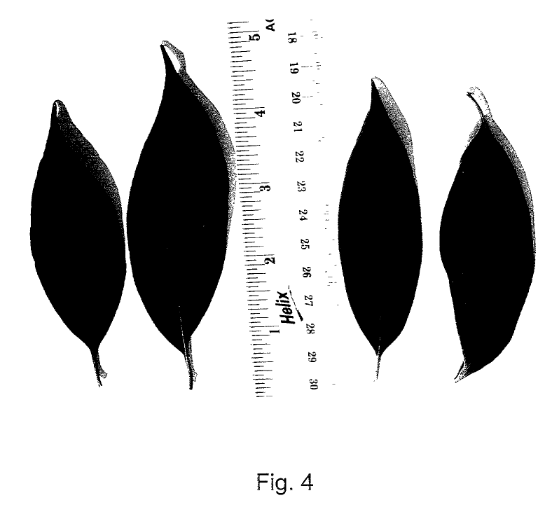

### DETAILED BOTANICAL DESCRIPTION

The following observations, measurements and values were obtained from the original ten to twelve year old parent plant obtained in 1993, while in bloom, growing on the property of the Inventor in Greenwood, S.C. These characteristics are known to apply to plants of the variety grown under similar conditions of soil and climate. In the following description, color references are made to The Royal Horticultural Society Colour Chart (London, 1966), except where general terms of ordinary dictionary significance are used.

Botanical classification: Cornus angustata (Chun) T. R. Dudley cultivar ‘Elsbry’.

Parentage: A seedling of Cornus angustata of unknown origin.

Propagation: Chip or T budding during August and September on Cornus florida or Cornus kousa seedling understocks.

Plant description:

Plant form and growth habit.—Perennial evergreen, multi-stemmed tree, vase shaped when young, becoming more rounded and spreading with age.

Vigor.—Slow to medium in early years becoming more vigorous with age.

Plant size.—The 10-12 year old parent plant attained a height and width of approximately 4 meters. Mature trees will achieve a height and width of approximately 8-10 meters.

Branching habit.—Moderate, branch crotch angles of about 45° to main trunk.

Main stem or truck description.—Diameter approximately 15 cm. with bark texture being smooth. Bark color being grayed-green close to color 197C, with prominent gray-brown color of approximately 199D, lenticels.

Lateral branch description.—Diameter approximately 7 cm. with the texture being smooth. The color is gray-brown being close to color 199B with prominent lenticels.

Foliaged Description:

Arrangement.—Simple, opposite.

Length.—Approximately 8 to 12 cm.

Width.—Approximately 2 to 3 cm.

Petiole length.—Approximately 1 cm.

Shape.—Elliptic-ovate.

Apex.—Acuminate.

Base.—Cuneate.

Margin.—Entire.

Texture.—Upper surface being glossy, nearly glabrous with numerous minute white hairs pressed against the surface. The lower surface being dulled, with numerous minute white hairs pressed against the surface.

Color.—Upper surface being uniform dark green of a color close to 139A with a prominent and paler mid-rib having a color close to yellow-green 145A. The lower surface being glaucous with gray-green color close to 191B with a prominent grayed-yellow mid-rib of a color close to 160C.

Flower description:

Natural flowering season.—Early to midsummer.

Inflorescence arrangement.—Inconspicuous flowers are arranged in dense, tight rounded terminal cymes on 4-6 cm. long upright peduncules and subtended by a corolla-like involucre of four showy bracts.

Inflorescence diameter.—Approximately 6 to 7 cm.

Involucral bracts.—Length of approximately 3-4 cm. with a width of approximately 2 to 2½ cm. The shape being ovate to obovate with a pointed apex. Young bracts being bi-colored with the basal half grayed-green close to color 193A, and the terminal half being green-white, close to color 157B. Eventually entire bract is green-white, close to color 157B.

Fruit description:

Type.—Drupe.

Shape.—Globose, on a 6 cm. long pendulous peduncle.

Diameter.—Approximately 2 cm.

Color.—Orange-red close to color 33A.

Disease resistance: Resistance to disease common to related Dogwoods such as powdery mildew and leaf and stem anthracnose.

What is claimed is:

1. A new and distinct cultivar of evergreen Dogwood tree named ‘Elsbry’, substantially as herein shown and described, characterized particularly as to novelty by the unique combination of its vigorous growth rate, flowering at a young age, prolific flower production on an annual basis, blooms 2-3 weeks later and over a longer period than other Cornus kousa selections, fruits extend ornamental interest into the early fall season, increased disease resistance, and good cold hardiness.
